SEQUENCE LISTING (36 sequences)

SEQ ID NO: 1 - DNA, 741 bp, Artificial Sequence
  acetoacetyl CoA reductase
atgcagagccgcgtggccctggtgaccggcggcaccggcggcatcggcacctcgatcatccagcgcctggcccagaccggccaccgcgtggccaccaactaccgcgacgaggaccgcgcgcgcgcctggcaggagaagatgcgcgcggccggcgtggaggtggccctggccccgggcgacgtgtcgagcccggagcaggcccaggccctggtggagcagatccagcgcgacctgggcccgatcgaggtgctggtgaacaacgccggcatcacccgcgactcgaccttccaccgcatgaccccgctgcagtggaacgaggtgatcggcaccaacctgaacagctgcttcaacgtgacccgcccggtgatcgagggcatgcgcgagcgcaagtggggccgcatcgtgcagatctcgagcatcaacggcctgaagggccagtacggccaggccaactacgcggccgccaaggcgggcatgcacggcttcaccatcagcctggcccgcgagaacgcccgcttcggcgtgaccgtgaacaccgtgtcgccgggctacgtggccaccgacatggtgatgagcgtgccggaggaagtgcgcgcgaagatcgtggcggagatcccgaccggccgcctgggcaccccgaacgagatcgcgtacgccgtgtcgttcctggtggcggaggaagccgcctggatcaccggcagcaacctggacatcaacggtggccaccacatgggctggtaa

SEQ ID NO: 2 - DNA, 741 bp, Artificial Sequence
  acetoacetyl-CoA reductase
atgcagaagctggccctgatcaccggcagcaagggcggcatcggctcggccatcaccgagaagctggtgcaggacggcttccgcatcatcgcgacctacttcaccggcaactacgagtgcgccaaggagtggttcgacgagaagggcttcagcgaggaccaggtgaccctgttcgagctggacgtgaccgacgcggacagctgccgcgagcgcctgaccaagctgctggagaacgagggcaccgtggacgtgctggtgaacaacgcgggcatcacccgcgactgcaccttcaagcgcatgaccgccgagcagtggaacgacgtgatcaacaccaacctgaacagcgtgttcaacgtgacccagccgctgttcgcggccatgtgcgagaagggcggcggccgcatcatcaacatctcgagcgtgaacggcctgaagggccagttcggccagaccaactactcggccgcgaaggcgggcatgatcggcttcagcaaggccctggccttcgagggcgcccgcagcggcgtgaccgtgaacgtggtggccccgggctacaccggcaccccgatggtgcaggccatccgccaggacgtgctggacagcatcatcgaaaccgtgccgatgaagcgcctggccaccccggaggagatcgccagcgccgtggcctacctggcctcggacgccggcgcctacatcaccggcgaaaccctgagcgtgaacggcggcctgtacatgcagtaa

SEQ ID NO: 3 - DNA, 816 bp, Artificial Sequence
  acetacetyl-CoA reductase
atgaccaccgccgcggccgagaagcgcgcggccaccaactcgggctcggagccggtgcgcgaggccgtgccgccgctgaagcagcgcatcgccttcgtgtcgggcggcatgggcggcatcggcagcgcggtgtgccgtcgcctggtgcgcaacggcgcccgcgtggtggcgggctgcctgccgggctacgagaagaaggacgagtggatcgccaagatgcgcggcgagggcctgcaggtgcacgccgccgagggcgacgtggacgactacgagtcgtgcgcggacatgttctaccagatcggcagcgtgatcggcccggtggacatcctggtgaacaacgccggcatcacccgcgacggcatgttcaagcgcatgagcccggccgactggtacgccgtgatcaacaccaacctgaacagcgtgttcaacgtgacccgccaggtgatcgagggcatgatggagcgcggctggggccgcatcatcaacatctcgagcgtgaacgcgctgaagggccagttcggccagaccaactacagcgccgccaaggcgggcatgcacggcttcagcaaggcgctggcccaggaagtggtgaagaagggcgtgaccgtgaacaccgtgtcgccgggctacgtggaaaccgacatggtgcgcgccatgaagccggaggtgctgcagagcatcgtggagcagatcccgatgggccgcttcgcccagccggaggagatcgccagcctggtggcctacctggcctcggaggaagccggctacatcaccggcgccaacatctcgatcaacggcggcctgcacatggtgtaa

SEQ ID NO: 4 - DNA, 747 bp, Artificial Sequence
  acetoacetyl-CoA reductase
atggcgaaccaggccccggtggcctgggtgaccggcggcaccggcggcatcggcaccgccatctgccgctcgctggcggacgccggctacctggtggtggccggctaccacaacccggagaaggccaagacctggctggccagccagcaggccgacggctacaacaacatcgccctgagcggcgtggacctggcggaccaccaggcctgcctgcagggcgcccagcagatccaggagcagcacggcccgatctcggtgctggtgaactgcgcgggcatcacccgcgacggcaccatgaagaagatgagctacgagcagtggtacgaggtgatcgacaccaacctgaactcggtgttcaacacctgccgcagcgtgatcgagatgatgctggagaacggctacggccgcatcatcaacatctcgagcatcaacggccgcaagggccagttcggccaggccaactacgcggccgccaaggccggcatgcacggcctgaccatgtcgctggcccaggaaaccgcgaccaagggcatcaccgtgaacaccgtgagcccgggctacatcgccaccgacatgatcatgaacatcccggagaaggtgcgcgaggccatccgcgaaaccatcccggtgaagcgctacggcaccccggaggagatcggccgcctggtgaccttcatcgccgacaaggagagcggcttcatcaccggcgcgaacttcgacatcaacggtggccagttcatgggctaa

SEQ ID NO: 5 - DNA, 741 bp, Artificial Sequence
  acetoacetyl-CoA reductase
atgtcgcgcgtggccctggtgaccggcggcatgggcggcctgggcgaggcgatctgcatcaagatggcggccctgggctaccaggtggtgaccacctactcgccgggcaacaagacctggaaggagtggctggacagcatgaacaagatgggcttcggcttccgcgcctacccgtgcgacgtggccgactacgacagcgccaccgcctgcgtggccctgatcgtgaaggaagtgggcccggtggacgtgctggtgaacaacgcgggcatcacccgcgacatgaccttcaagaagatggacaagatcaactgggacgccgtggtggcgaccaacctggacagcaccttcaacatgaccaagcaggtgtgcgacggcatgctggagcgcggctggggccgcatcgtgaacatctcgtcggtgaacggccagaagggcgccttcggccagaccaactacagcgcggccaaggccggcatgcacggcttcaccaaggccctggccctggaggtggcccgcaagggcgtgaccgtgaacaccatcagcccgggctacatcggcaccaagatggtgatggcgatcccgtcggacgtgctggagagcaagatcatcccgcagatcccgatgggccgcctgggcaagccggaggaagtggcgggcctggtggcctacctggccagcgacgaggcggcgttcctgaccggcgccaacatcgcgatcaacggtggccagcacatgagctaa

SEQ ID NO: 6 - DNA, 744 bp, Artificial Sequence
  acetoacetyl-CoA reductase
atgacccagcgcatcgccatcgtgaccggcgccaacggcggcctgggcgagagcatgtgcaaggccctggccgagcagggccgcaaggtggtgggcaccttcgtgccgggccacgacgcggccgcggccagctggcaggaagagatgaagcagcagggcttcgacatcaccatgaaggccgtggacgtgtcggacttcaacgactgcaagcgcctgctggaggagatcgagtcgagcgagggcagcgtggacatcctggtgaacaacgcgggcatcacccgcgacgcgccgctgaagaagatgcagccggagcagtggcaggcggtgatcggcaccaacctgaactcgatgttcaacatgagccagccgatcttcgaggccatgtgcaaccgcggctggggccgcatcgtgaacatctcgagcctgaacggcgagaagggccagttcggccaggccaactacagcgcggccaaggcgggcatctacggcttcaccaaggccatcgcgcaggaaggcgccaagaagggcgtgaccgtgaactcggtgagcccgggctacatcgacaccccgatggtgcgccaggtgccggagaacgtgctgaacagcatcgtgggcggcatcccggtgggccgcctgggccagccggaggagatcgcccgcgcggtgtcgttcctgaccgcggacgacgccggcttcatcaccggcaccaacatcagcgtgaacggtggccagtacatggcgtaa

SEQ ID NO: 7 - DNA, 741 bp, Artificial Sequence
  acetoacetyl-CoA reductase
atgtcgcgcgtggccctggtgaccggcggcatgggcggcctgggcgaggcgatctgcatcaagctggccgcgctgggctacaaggtggtgaccacccacagcccgggcaacaccaaggcggccgagtggctgctgaccatgaacaacatgggctacggcttcaaggcctacccgtgcgacgtgagcgacttcgactcgagcaaggcgtgcgtggaaaccgtgacccgcgaagtgggcccggtggacgtgctggtgaacaacgccggcatcacccgcgacatgaccttcaagaagatgaccaaggccgactgggacgcggtgatcggcaccaacctggactcggtgttcaacatgaccaagcaggtgatggacggcatggtggagcgtcgctggggccgcgtgatcaacgtgtcgtcggtgaacggccagaagggcgccttcggccagaccaactactcggcggccaaggccggcatgcacggcttcaccaaggccctggccctggaggtggcccgcaacggcgtgaccgtgaacaccatctcgccgggctacatcggcaccaagatggtgatggccatcccgcaggagatcctggacagcaagatcctgccgcagatcccgctggcccgcctgggcaagccggaggagatcgcgggcctggtggcctacctgagctcggaggaagccgccttcgtgaccggcgcgaacatcagcatcaacggtggccagcacatgttctaa

SEQ ID NO: 8 - DNA, 2887 bp, Artificial Sequence
  phaC gene
cgggcgtgcccatgatgtagagcaccagcgccaccggcgccatgccatacatcaggaaggtggcaacgcctgccaccacgttgtgctcggtgatcgccatcatcagcgccacgtagagccagccaatggccacgatgtacatcaaaaattcatccttctcgcctatgctctggggcctcggcagatgcgagcgctgcataccgtccggtaggtcgggaagcgtgcagtgccgaggcggattcccgcattgacagcgcgtgcgttgcaaggcaacaatggactcaaatgtctcggaatcgctgacgattcccaggtttctccggcaagcatagcgcatggcgtctccatgcgagaatgtcgcgcttgccggataaaaggggagccgctatcggaatggacgcaagccacggccgcagcaggtgcggtcgagggcttccagccagttccagggcagatgtgccggcagaccctcccgctttgggggaggcgcaagccgggtccattcggatagcatctccccatgcaaagtgccggccagggcaatgcccggagccggttcgaatagtgacggcagagagacaatcaaatcatgagccaaccatcttatggcccgctgttcgaggccctggcccactacaatgacaagctgctggccatggccaaggcccagacagagcgcaccgcccaggcgctgctgcagaccaatctggacgatctgggccaggtgctggagcagggcagccagcaaccctggcagctgatccaggcccagatgaactggtggcaggatcagctcaagctgatgcagcacaccctgctcaaaagcgcaggccagccgagcgagccggtgatcaccccggagcgcagcgatcgccgcttcaaggccgaggcctggagcgaacaacccatctatgactacctcaagcagtcctacctgctcaccgccaggcacctgctggcctcggtggatgccctggagggcgtcccccagaagagccgggagcggctgcgtttcttcacccgccagtacgtctctgccatggcccccagcaacttcctggccaccaaccccgagctgctcaagctgaccctggagtccggcggccagaacctggtgcgcggactggccctcttggccgaggatctggagcgcagcgccgatcagctcaacatccgcctgaccgacgaatccgccttcgagctcgggcgggatctggccctgaccccgggccgggtggtgcagcgcaccgagctctatgagctcattcagtacagcccgactaccgagacggtgggcaagacacctgtgctgatagtgccgcccttcatcaacaagtactacatcatggacatgcggccccagaactccctggtcgcctggctggtcgcccagggccagacggtattcatgatctcctggcgcaacccgggcgtggcccaggcccaaatcgatctcgacgactacgtggtggatggcgtcatcgccgccctggacggcgtggaggcggccaccggcgagcgggaggtgcacggcatcggctactgcatcggcggcaccgccctgtcgctcgccatgggctggctggcggcgcggcgccagaagcagcgggtgcgcaccgccaccctgttcactaccctgctggacttctcccagcccggggagcttggcatcttcatccacgagcccatcatagcggcgctcgaggcgcaaaatgaggccaagggcatcatggacgggcgccagctggcggtcagcttcagcctgctgcgggagaacagcctctactggaactactacatcgacagctacctcaagggtcagagcccggtggccttcgatctgctgcactggaacagcgacagcaccaatgtggcgggcaagacccacaacagcctgctgcgccgtctctacctggagaaccagctggtgaagggggagctcaagatccgcaacacccgcatcgatctcggcaaggtgaagacccctgtgctgctggtgtcggcggtggacgatcacatcgccctctggcagggcacctggcagggcatgaagctgtttggcggggagcagcgcttcctcctggcggagtccggccacatcgccggcatcatcaacccgccggccgccaacaagtacggcttctggcacaacggggccgaggccgagagcccggagagctggctggcaggggcgacgcaccagggcggctcctggtggcccgagatgatgggctttatccagaaccgtgacgaagggtcagagcccgtccccgcgcgggtcccggaggaagggctggcccccgcccccggccactatgtcaaggtgcggctcaaccccgtgtttgcctgcccaacagaggaggacgccgcatgacgcttgcatgagtgccggcgtgcgtcatgcacggcgccggcaggcctgcaggttccctcccgtttccattgaaaggactacacaatgactgacgttgtcatcgtatccgccgcccgcaccgcggtcggcaagtttggcggctcgctggccaagatcccggcaccggaactgggtgccgtggtcatcaaggccgcgctggagcgcgccggcgtcaagccggagcaggtgagcgaagtcatcatgggccaggtgctgaccgccggttcgggccagaaccccgcacgccaggccgcgatcaaggccggcctgccggcgatggtgccggccatgaccatcaacaaggtgtgcggctcgggcctgaaggccgtgatgctggccgccaacgcgatcatggcgggcgacgccgagatcgtggtggccggcggccaggaaaacatgagcgccgccccgcacgtgctgccgggctcgcgcgatggtttccgcatgggcgatgccaagctggtcgacaccatg

SEQ ID NO: 9 - protein, 246 aa, Artificial Sequence
  acetocetyl-CoA reductase
MQSRVALVTGGTGGIGTSIIQRLAQTGHRVATNYRDEDRARAWQEKMRAAGVEVALAPGDVSSPEQAQALVEQIQRDLGPIEVLVNNAGITRDSTFHRMTPLQWNEVIGTNLNSCFNVTRPVIEGMRERKWGRIVQISSINGLKGQYGQANYAAAKAGMHGFTISLARENARFGVTVNTVSPGYVATDMVMSVPEEVRAKIVAEIPTGRLGTPNEIAYAVSFLVAEEAAWITGSNLDINGGHHMGW

SEQ ID NO: 10 - protein, 246 aa, Artificial Sequence
  acetoacetyl-CoA reductase
MQKLALITGSKGGIGSAITEKLVQDGFRIIATYFTGNYECAKEWFDEKGFSEDQVTLFELDVTDADSCRERLTKLLENEGTVDVLVNNAGITRDCTFKRMTAEQWNDVINTNLNSVFNVTQPLFAAMCEKGGGRIINISSVNGLKGQFGQTNYSAAKAGMIGFSKALAFEGARSGVTVNVVAPGYTGTPMVQAIRQDVLDSIIETVPMKRLATPEEIASAVAYLASDAGAYITGETLSVNGGLYMQ

SEQ ID NO: 11 - protein, 271 aa, Artificial Sequence
  acetoacetyl-CoA reductase
MTTAAAEKRAATNSGSEPVREAVPPLKQRIAFVSGGMGGIGSAVCRRLVRNGARVVAGCLPGYEKKDEWIAKMRGEGLQVHAAEGDVDDYESCADMFYQIGSVIGPVDILVNNAGITRDGMFKRMSPADWYAVINTNLNSVFNVTRQVIEGMMERGWGRIINISSVNALKGQFGQTNYSAAKAGMHGFSKALAQEVVKKGVTVNTVSPGYVETDMVRAMKPEVLQSIVEQIPMGRFAQPEEIASLVAYLASEEAGYITGANISINGGLHMV

SEQ ID NO: 12 - protein, 248 aa, Artificial Sequence
  acetoacetyl-CoA reductase
MANQAPVAWVTGGTGGIGTAICRSLADAGYLVVAGYHNPEKAKTWLASQQADGYNNIALSGVDLADHQACLQGAQQIQEQHGPISVLVNCAGITRDGTMKKMSYEQWYEVIDTNLNSVFNTCRSVIEMMLENGYGRIINISSINGRKGQFGQANYAAAKAGMHGLTMSLAQETATKGITVNTVSPGYIATDMIMNIPEKVREAIRETIPVKRYGTPEEIGRLVTFIADKESGFITGANFDINGGQFMG

SEQ ID NO: 13 - protein, 246 aa, Artificial Sequence
  acetoacetyl-CoA reductase
MSRVALVTGGMGGLGEAICIKMAALGYQVVTTYSPGNKTWKEWLDSMNKMGFGFRAYPCDVADYDSATACVALIVKEVGPVDVLVNNAGITRDMTFKKMDKINWDAVVATNLDSTFNMTKQVCDGMLERGWGRIVNISSVNGQKGAFGQTNYSAAKAGMHGFTKALALEVARKGVTVNTISPGYIGTKMVMAIPSDVLESKIIPQIPMGRLGKPEEVAGLVAYLASDEAAFLTGANIAINGGQHMS

SEQ ID NO: 14 - protein, 247 aa, Artificial Sequence
  acetoacetyl-CoA reductase
MTQRIAIVTGANGGLGESMCKALAEQGRKVVGTFVPGHDAAAASWQEEMKQQGFDITMKAVDVSDFNDCKRLLEEIESSEGSVDILVNNAGITRDAPLKKMQPEQWQAVIGTNLNSMFNMSQPIFEAMCNRGWGRIVNISSLNGEKGQFGQANYSAAKAGIYGFTKAIAQEGAKKGVTVNSVSPGYIDTPMVRQVPENVLNSIVGGIPVGRLGQPEEIARAVSFLTADDAGFITGTNISVNGGQYMA

SEQ ID NO: 15 - protein, 246 aa, Artificial Sequence
  acetoacetyl-CoA reductase
MSQKIALVTGAMGGLGTAICQSLARDGMKVVANCLPGFDQKEGWLAAQRDLGFNFIAAEGDVSDYDSCAAMVARIEAEVGPVDVLVNNAGITRDKFFPKMQKAQWDAVINTNLNSLFNVTHHVSAKMAERGWGRIISISSVNGVKGQAGQTNYSAAKAGVLGFTKALAAELATKGVTVNAVAPGYVGTEMVMAIRDDIRQGIIDTIPMGRLGRPDEIGDLCAYLASDKAAYITGATININGGLHMC

SEQ ID NO: 16 - protein, 593 aa, Artificial Sequence
  PHA polymerase
MSQPSYGPLFEALAHYNDKLLAMAKAQTERTAQALLQTNLDDLGQVLEQGSQQPWQLIQAQMNWWQDQLKLMQHTLLKSAGQPSEPVITPERSDRRFKAEAWSEQPIYDYLKQSYLLTARHLLASVDALEGVPQKSRERLRFFTRQYVSAMAPSNFLATNPELLKLTLESGGQNLVRGLALLAEDLERSADQLNIRLTDESAFELGRDLALTPGRVVQRTELYELIQYSPTTETVGKTPVLIVPPFINKYYIMDMRPQNSLVAWLVAQGQTVFMISWRNPGVAQAQIDLDDYVVDGVIAALDGVEAATGEREVHGIGYCIGGTALSLAMGWLAARRQKQRVRTATLFTTLLDFSQPGELGIFIHEPIIAALEAQNEAKGIMDGRQLAVSFSLLRENSLYWNYYIDSYLKGQSPVAFDLLHWNSDSTNVAGKTHNSLLRRLYLENQLVKGELKIRNTRIDLGKVKTPVLLVSAVDDHIALWQGTWQGMKLFGGEQRFLLAESGHIAGIINPPAANKYGFWHNGAEAESPESWLAGATHQGGSWWPEMMGFIQNRDEGSEPVPARVPEEGLAPAPGHYVKVRLNPVFACPTEEDA

SEQ ID NO: 17 - DNA, 169 bp, Artificial Sequence
  promoter
atgcctccacaccgctcgtcacatcctgttgcgttcactggaatcccagtatctaatttgacctgcgagcaagctgtcaccggatgtgctttccggtctgatgagtccgtgaggacgaaacagcctctacaaataattttgtttaactgactgaattcatgggacaagc

SEQ ID NO: 18 - DNA, 20 bp, Artificial Sequence
  pK-R primer
gcagacttggccgggtacca

SEQ ID NO: 19 - DNA, 19 bp, Artificial Sequence
  pK-F primer
caccgctcgtcacatcctg

SEQ ID NO: 20 - DNA, 40 bp, Artificial Sequence
  phaCH1-F primer
tggtacccggccaagtctgcgggcgtgcccatgatgtaga

SEQ ID NO: 21 - DNA, 42 bp, Artificial Sequence
  phaCH1-R primer
tgagacccaaggtctccatgatttgattgtctctctgccgtc

SEQ ID NO: 22 - DNA, 38 bp, Artificial Sequence
  phaCH2-F primer
ggagaccttgggtctcagtgacgcttgcatgagtgccg

SEQ ID NO: 23 - DNA, 39 bp, Artificial Sequence
  phaCH2-R
caggatgtgacgagcggtgcatggtgtcgaccagcttgg

SEQ ID NO: 24 - DNA, 11 bp, Artificial Sequence
  primer
gtgaagagacc

SEQ ID NO: 25 - DNA, 21 bp, Artificial Sequence
  FphaCH1-F primer
tggtctggctggcggactgag

SEQ ID NO: 26 - DNA, 21 bp, Artificial Sequence
  phaCH2-R primer
ggcgaactcatcctgcgcctc

SEQ ID NO: 27 - DNA, 24 bp, Artificial Sequence
  pKCH-C R primer
catgatttgattgtctctctgccg

SEQ ID NO: 28 - DNA, 20 bp, Artificial Sequence
  pKCH-C F primer
gtgacgcttgcatgagtgcc

SEQ ID NO: 29 - DNA, 39 bp, Artificial Sequence
  phaB F primer
agagagacaatcaaatcatgactcagcgcattgcgtatg

SEQ ID NO: 30 - DNA, 40 bp, Artificial Sequence
  phaB R primer
ggcactcatgcaagcgtcactcagcccatatgcaggccgc

SEQ ID NO: 31 - DNA, 37 bp, Artificial Sequence
  phaJ-H1 Fp primer
tggtacccggccaagtctgttcgacggcgtcttcgtt

SEQ ID NO: 32 - DNA, 38 bp, Artificial Sequence
  phaJ-H1 Rp primer
cgagcggtgtggaggcatctattcagtcagggatgcct

SEQ ID NO: 33 - DNA, 62 bp, Artificial Sequence
  phaJ-H2 Fp primer
ctacaaataattttgtttaactgactgaattcatgggacaagcatgaagacctacgagaaca

SEQ ID NO: 34 - DNA, 41 bp, Artificial Sequence
  phaJ-H2 Rp primer
cttgaagacgaaagggcctcgtggcgccttatggaaatcag

SEQ ID NO: 35 - DNA, 18 bp, Artificial Sequence
  phaJ Fp primer
atgcctccacaccgctcg

SEQ ID NO: 36 - DNA, 26 bp, Artificial Sequence
  phaJ Rp primer
ttaaacaaaattatttgtagaggctg

(19) United States
(12) Patent Application Publication
(10) Pub. No.: US 2024/0263130 A1
(43) Pub. Date: Aug. 8, 2024

(54) ENGINEERED MICROORGANISMS EXPRESSING ACETOACETYL- COA REDUCTASE VARIANTS AND METHODS FOR INCREASING PROPORTION OF 3-HYDROXYHEXANOIC ACID IN PHA

(71) Applicant: SHENZHEN BLUEPHA BIOSCIENCES CO., LTD., Guangdong (CN)

(72) Inventors: Jin YIN, Guangdong (CN); Yu WANG, Guangdong (CN); Jiajia LI, Guangdong (CN); Jie HOU, Guangdong (CN); Liang ZOU, Guangdong (CN); Zixian CHEN, Guangdong (CN)

(73) Assignee: SHENZHEN BLUEPHA BIOSCIENCES CO., LTD., Guangdong (CN)

(21) Appl. No.: 18/031,556

(22) PCT Filed: Jun. 28, 2022
(86) PCT No.: PCT/CN2022/101796

§ 371 Date: Apr. 12, 2023

(30) Foreign Application Priority Data
Apr. 6, 2022 (CN) 202210353444.8

(51) Int. Cl. C12N 1/20; C12N 9/04; C12N 9/10; C12N 9/88; C12N 15/74; C12N 15/90; C12P 7/625; C12R 1/01

(52) U.S. Cl. CPC C12N 1/20; C12N 9/0006; C12N 9/1029; C12N 9/88; C12N 15/74; C12N 15/90; C12P 7/625; C12N 2510/00; C12R 2001/01; C12Y 101/01036; C12Y 402/01119

(57) ABSTRACT

The present invention relates to the technical field of microorganisms, and specifically to engineered microorganisms expressing acetoacetyl-CoA reductase variants and methods for increasing the proportion of 3-hydroxyhexanoic acid in PHA. The acetoacetyl-CoA reductase variants and their coding genes provided by the present invention can significantly increase the proportion of 3-hydroxyhexanoic acid in PHA produced by strains; the proportion of 3-hydroxyhexanoic acid in PHA produced by the engineered Ralstonia eutropha constructed utilizing the acetoacetyl-CoA reductase variants and their coding genes provided by the present invention is significantly increased, which provides new genes and strain resources for strains producing poly(3-hydroxybutyrate-co-3-10 the development of engineered hydroxyhexanoate) with high proportion of 3-hydroxyhexanoic acid.

### CROSS REFERENCE TO RELATED APPLICATIONS

This application is a national stage application filed under 35 U.S.C. § 371 of International Patent Application No. PCT/CN2022/101796, filed Jun. 28, 2022, which claims the benefit of CN202210353444.8 filed Apr. 6, 2022.

### INCORPORATION OF MATERIAL OF ASCII TEXT SEQUENCE LISTING BY REFERENCE

The sequence listing submitted herewith as a text file named “CNKH-1041US_SUBSTITUTE_SEQUENCE_LISTING” created on Apr. 10, 2023, which is 37,000 bytes in size, is hereby incorporated by reference in its entirety.

### TECHNICAL FIELD

The present invention relates to the technical field of microorganisms, specifically, to engineered microorganisms expressing acetoacetyl-CoA reductase variants and methods for increasing the proportion of 3-hydroxyhexanoic acid in PHA.

### BACKGROUND ART

Polyhydroxyalkanoates (PHAs) are a class of polymers synthesized by microorganisms with renewable and degradable characteristics and multi-material properties, which have a wide range of applications in the fields of medicine, materials and environmental protection. PHAs are widely present in microbial cells, mainly acting as carbon sources and energy storage carriers. According to different monomer types and polymerization modes, PHAs have a series of material properties with diversity from hard and brittle hard plastic to soft elastomer. Polyhydroxy butyrate (PHB, or P(3HB)), one of the PHAs, is a bacterially produced complex biopolymer, as a thermoplastic, has mechanical and physical properties comparable to those of conventional plastics such as polystyrene. However, due to its highly crystalline nature, P(3HB) is more brittle and harder than most conventional plastics, to the extent that it is not practical as a substitute for commercial plastics. Therefore, it is necessary to produce a copolymer containing a combination of 3HB and longer chain monomers to achieve improvement in thermal and physical properties. Poly(3-hydroxy butyrate-co-3-hydroxyhexanoate) (P(3HB-co-3HHx)) is a hybrid chain-length copolymer that is more flexible than the P(3HB) homopolymer, where the 3HHx (3-hydroxyhexanoate) monomer fraction largely determines the properties of the polymer. P(3HB-co-3HHx) copolymers with a high 3HHx molar fraction (referred to as H ratio) can exhibit properties similar to those of common commercial plastics such as low density polyethylene (LDPE). Therefore, it is of great significance to increase the proportion of 3HHx in 3HB-co-3HHx produced by the microorganisms.

Ralstonia eutropha (also known as Cupriavidus necator) is an important model bacterium for the study of PHA synthesis and is the most studied strain for PHB production. Ralstonia eutropha can accumulate PHB when carbon is in excess and nitrogen is deficient, while PHB synthesis is compromised when other intracellular carbon sources are metabolized vigorously.

### SUMMARY OF THE INVENTION

The purpose of the present invention is to provide an acetoacetyl-CoA reductase variant and its coding gene capable of increasing the proportion of 3-hydroxyhexanoic acid in PHA produced by microorganisms, and engineered microorganisms for PHA production.

Specifically, the present invention provides the following technical solutions:

In a first aspect, the present invention provides an acetoacetyl-CoA reductase variant with amino acid sequence as represented by any one of SEQ ID NOs. 9-15.

In a second aspect, the present invention provides gene variants encoding the acetoacetyl-CoA reductase with nucleotide sequences as represented by any one of SEQ ID NOs. 1-7.

The present invention demonstrates experimentally that each of the above gene variants is capable of significantly increasing the proportion of 3-hydroxyhexanoic acid in PHA produced by the strains.

In a third aspect, the present invention provides biological material containing the above gene variants, the biological material being an expression cassette, a vector or a host cell.

In some embodiments of the present invention, the expression cassette containing the gene variants described above is obtained by operably linking a promoter, the gene variant described above, and a terminator. Depending on the need of expression and the upstream and downstream sequences of the expression cassette, the expression cassette may also not contain a terminator or may contain other transcription and translation regulatory elements such as enhancers.

In some embodiments of the present invention, the vectors containing the gene variants described above are plasmid vectors, which include replication-competent vectors and non-replication-competent vectors. The vectors carrying the nucleic acid molecules described above are not limited to plasmid vectors, but may also be vectors such as phages, viruses, and the like.

In some embodiments of the present invention, cells of Escherichia coli and Ralstonia eutropha containing the above gene variants, expression cassettes or vectors are provided, but the type of host cells is not limited to this, and can be any microbial cells or animal cells that can be used for protein expression.

In a fourth aspect, the present invention provides use of an acetoacetyl-CoA reductase variant or its coding gene in increasing the proportion of 3-hydroxyhexanoic acid in PHA produced by engineered microorganisms, the acetoacetyl-CoA reductase variant having an amino acid sequence as represented by any one of SEQ ID NOs. 9-15.

Based on the amino acid sequences of the acetoacetyl-CoA reductase variants provided above and the codon rules, a person skilled in the art is able to obtain the sequences of genes encoding the acetoacetyl-CoA reductase variants described above. The sequences of genes encoding the same amino acid sequence are not unique, but all genes capable of encoding the acetoacetyl-CoA reductase variants described above are within the scope of protection of the present invention.

In some embodiments of the present invention, the nucleotide sequence of the coding gene is represented by any one of SEQ ID NOs. 1-7.

The above use can be achieved by any one or more of the following ways:
(1) introducing a plasmid comprising a gene encoding the acetoacetyl-CoA reductase variant:
(2) inserting one or more copies of the gene encoding the acetoacetyl-CoA reductase variant into the genome.

In some embodiments of the present invention, the genomic in situ phaB gene of the microorganism is not inactivated.

In some embodiments of the present invention, the genomic in situ phaB gene of the microorganism is retained intact.

In the above use, the engineered microorganism further includes one or more of the following modifications:
(1) expression of a PHA polymerase variant capable of synthesizing poly(3-hydroxy butyrate-co-3-hydroxyhexanoate);
(2) enhanced expression and/or enzyme activity of (R)-enoyl-CoA hydratase.

In some embodiments of the present invention, the PHA polymerase variant capable of synthesizing poly(3-hydroxy butyrate-co-3-hydroxyhexanoate) contains a mutation of asparagine to serine at position 149 and a mutation of aspartate to glycine at position 171 compared to the original PHA polymerase.

In some embodiments of the present invention, the amino acid sequence of the PHA polymerase variant is represented by SEQ ID NO. 16.

In some embodiments of the present invention, the enhanced expression and/or enzyme activity of (R)-enoyl-CoA hydratase is achieved by initiating the transcription of the gene encoding (R)-enoyl-CoA hydratase in the genome with the promoter represented by SEQ ID NO. 17.

In the above use, the microorganism is preferably Ralstonia eutropha, E. coli or Halomonas.

In some embodiments of the present invention, the increase in the proportion of 3-hydroxyhexanoic acid in PHA produced by the engineered microorganism is an increase in the proportion of 3-hydroxyhexanoic acid in PHA produced by the engineered microorganism using lipids as carbon sources.

In some embodiments of the present invention, the increase in the proportion of 3-hydroxyhexanoic acid in PHA produced by the engineered microorganism is an increase in the proportion of 3-hydroxyhexanoic acid in PHA produced by the engineered microorganism using vegetable oils as carbon sources.

The vegetable oils include a mixture selected from one or more of palm oil, peanut oil, soy bean oil, flax oil, rapeseed oil, castor oil, and corn oil. In a fifth aspect, the present invention provides the application of an acetoacetyl-CoA reductase variant or its coding gene or biological material containing the coding gene in the construction of microorganisms for the production of polyhydroxy fatty acid esters or their derivatives.

Based on the function that the acetoacetyl-CoA reductase variants provided by the present invention can increase the proportion of 3-hydroxyhexanoic acid in PHA produced by microorganisms, the genes encoding these variants and the biological materials containing these genes can be used in the construction of strains for the production of PHA.

In the above applications, the microorganisms are preferably Ralstonia eutropha, E. coli or Halomonas.

In some embodiments of the present invention, the gene represented by any one of SEQ ID NOs. 1-7 is introduced into Ralstonia eutropha to construct a PHA-producing strain.

In a sixth aspect, the present invention provides an engineered Ralstonia eutropha, which expresses the acetoacetyl-CoA reductase variant, the acetoacetyl-CoA reductase variant having an amino acid sequence as represented by any one of SEQ ID NOs. 9-15.

In the present invention, expression of the acetoacetyl-CoA reductase variant may be achieved by any one or more of the following methods:
(1) introducing a plasmid comprising a gene encoding the acetoacetyl-CoA reductase variant:
(2) inserting one or more copies of the gene encoding the acetoacetyl-CoA reductase variant into the genome.

In some embodiments of the present invention, the acetoacetyl-CoA reductase variant is expressed by inserting one copy of the gene encoding the acetoacetyl-CoA reductase variant into the genome.

In some embodiments of the present invention, the acetoacetyl-CoA reductase variant is inserted at the position where the phaC gene is located in the genome.

In some embodiments of the present invention, the gene encoding the acetoacetyl-CoA reductase variant is represented by any one of SEQ ID NOs. 1-7. The sequences of these coding genes are those capable of achieving efficient and correct expression of the acetoacetyl-CoA reductase variant in Ralstonia eutropha obtained according to the codon preference of Ralstonia eutropha and combined with manual optimization and screening, the use of which facilitates the acetoacetyl-CoA reductase variant to better function in Ralstonia eutropha to increase the proportion of 3-hydroxyhexanoic acid in PHA.

In some embodiments of the present invention, the genomic in situ phaB gene of the microorganism is not inactivated.

In some embodiments of the present invention, the genomic in situ phaB gene of the microorganism is retained intact.

In some embodiments of the present invention, in order to facilitate synthesis of poly(3-hydroxy butyrate-co-3-hydroxy hexanoate), the engineered Ralstonia eutropha further includes one or more of the following modifications:
(1) expression of a PHA polymerase variant capable of synthesizing poly(3-hydroxy butyrate-co-3-hydroxyhexanoate;
(2) enhanced expression and/or enzyme activity of (R)-enoyl-CoA hydratase.

Wherein, the PHA polymerase variant capable of synthesizing poly(3-hydroxy butyrate-co-3-hydroxyhexanoate) can be made by mutating the amino acid sequence of the bacterial PHA polymerase to enable it to polymerize C6 fatty acid (3-hydroxyhexanoic acid), either by using a prior art PHA polymerase variant capable of polymerizing C6 fatty acid (3-hydroxyhexanoic acid), or by combining the mutation sites of prior art PHA polymerase variants capable of polymerizing poly(3-hydroxy butyrate-co-3-hydroxyhexanoate) to obtain new and more efficient PHA polymerase variants.

In some embodiments of the present invention, the PHA polymerase variant capable of synthesizing poly(3-hydroxy butyrate-co-3-hydroxyhexanoate) contains a mutation of asparagine to serine at position 149 and a mutation of aspartic acid to glycine at position 171 compared to the original PHA polymerase.

In some embodiments of the present invention, the amino acid sequence of the PHA polymerase variant is represented by SEQ ID NO. 16.

The above expression of a PHA polymerase variant capable of synthesizing poly(3-hydroxy butyrate-co-3-hydroxyhexanoate) is achieved by any one or more of the following ways:
(1) introducing a plasmid including a gene encoding the PHA polymerase variant capable of synthesizing poly(3-hydroxy butyrate-co-3-hydroxyhexanoate;
(2) inserting one or more copies of the gene encoding a variant of the PHA polymerase capable of synthesizing poly(3-hydroxy butyrate-co-3-hydroxyhexanoate) into the genome.

In some embodiments of the present invention, a PHA polymerase variant capable of synthesizing poly(3-hydroxy butyrate-co-3-hydroxyhexanoate) is expressed while the original PHA polymerase encoding gene of the genome is inactivated.

In some embodiments of the present invention, the gene encoding the PHA polymerase variant capable of synthesizing poly(3-hydroxy butyrate-co-3-hydroxyhexanoate) is inserted into the genome.

In some embodiments of the present invention, an expression plasmid containing a gene encoding a PHA polymerase variant capable of synthesizing poly(3-hydroxy butyrate-co-3-hydroxyhexanoate) is introduced.

In some embodiments of the present invention, the expression plasmid is a stable expression plasmid and stable expression of the plasmid is achieved by carrying in the plasmid a synthetic gene for a metabolite essential for the growth of the strain while inactivating the synthetic gene in the genome.

In some embodiments of the present invention, the plasmid containing the gene encoding the PHA polymerase variant represented by SEQ ID NO. 16 further contains the proC gene and the genomic proC gene of the engineered Ralstonia eutropha is inactivated.

The above enhancement of expression of the enzyme may be achieved by any one or more of the following ways (1)-(4):
(1) introducing a vector containing a gene encoding the enzyme:
(2) increasing the copy number of the gene encoding the enzyme in the genome:
(3) altering the sequence of transcription and/or translation regulatory elements (including promoters, and the like) of a gene encoding the enzyme in the genome:
(4) altering the nucleotide sequence of the gene encoding the enzyme.

The enhancement of the enzyme activity may be achieved by substitution, deletion or insertion of one or more amino acids of the enzyme.

In some embodiments of the present invention, the enhanced expression and/or enzyme activity of (R)-enoyl-CoA hydratase is achieved by initiating the transcription of the gene encoding (R)-enoyl-CoA hydratase in the genome with the promoter represented by SEQ ID NO. 17.

In some embodiments of the present invention, initiation of transcription of the gene encoding (R)-enoyl-CoA hydratase in the genome is achieved by inserting the promoter represented by SEQ ID NO. 17 in the intergenic region of the gene encoding genomic (R)-enoyl-CoA hydratase and its upstream gene.

The expression of (R)-enoyl-CoA hydratase is silenced in wild-type Ralstonia eutropha, and in the present invention, the promoter used to enhance the expression of (R)-enoyl-CoA hydratase and enzyme activity, is not limited to the promoter represented by SEQ ID NO. 17; all DNA fragments that can function as promoters in Ralstonia eutropha are capable of initiating transcription of (R)-enoyl-CoA hydratase and enhancing the expression and/or enzyme activity of (R)-enoyl-CoA hydratase.

In some embodiments of the present invention, the enhanced expression and/or enzyme activity of (R)-enoyl-CoA hydratase is achieved by initiating the transcription of the gene encoding (R)-enoyl-CoA hydratase in the genome with the promoter upstream of the (R)-enoyl-CoA hydratase gene in strain BPS-050 of Ralstonia eutropha.

The present invention demonstrates by experimental validation that the acetoacetyl-CoA reductase encoding gene variant of the present invention that function to increase the proportion of 3-hydroxy hexanoic acid in PHA produced by engineered microorganisms is not depend on the specific promoter sequence of the (R)-enoyl-CoA hydratase gene, but only on the ability of (R)-enoyl-CoA hydratase to be expressed in engineered microorganisms.

In some embodiments of the present invention, the engineered Ralstonia eutropha is obtained by modification with wild-type Ralstonia eutropha, Ralstonia eutropha H16 or Ralstonia eutropha BPS-050 as the original strain.

Among them, Ralstonia eutropha BPS-050 has been deposited in China General Microbiological Culture Collection Center (CGMCC for short, Address: Building 3, NO. 1 Courtyard, West Beichen Road, Chaoyang District, Beijing, Institute of Microbiology, Chinese Academy of Sciences, postal code: 100101) on Oct. 13, 2021, with the taxonomic designation of Ralstonia eutropha, a deposit number of CGMCC No. 23600.

In a seventh aspect, the present invention provides a library of engineered Ralstonia eutropha transformants, the library of transformants including at least 2 strains of the engineered Ralstonia eutropha described above.

In some embodiments of the present invention, the engineered Ralstonia eutropha in the library of transformants expresses an acetoacetyl-CoA reductase variant as represented by any one of SEQ ID NOs. 9-15, and each strain of the engineered Ralstonia eutropha in the library of transformants expresses an acetoacetyl-CoA reductase variant with a different amino acid sequence.

In some embodiments of the present invention, the library of transformants includes 2, 3, 4, 5, 6 or 7 strains of the engineered Ralstonia eutropha described above.

In an eighth aspect, the present invention provides a method of constructing the engineered Ralstonia eutropha, the method includes the step of modifying the Ralstonia eutropha to express the acetoacetyl-CoA reductase variant.

In some embodiments of the present invention, in order to promote the ability of the Ralstonia eutropha to synthesize poly(3-hydroxy butyrate-co-3-engineered hydroxyhexanoate), the method further includes one or more of the following modifications to the Ralstonia eutropha:
(1) expression of a PHA polymerase variant capable of synthesizing poly(3-hydroxybutyrate-co-3-hydroxyhexanoate);
(2) enhanced expression and/or enzyme activity of (R)-enoyl-CoA hydratase.

In some embodiments of the present invention, the amino acid sequence of the PHA polymerase variant capable of synthesizing poly(3-hydroxy butyrate-co-3-hydroxyhexanoate) is represented by SEQ ID NO. 16.

In some embodiments of the present invention, the method includes initiating the transcription of the gene encoding the genomic (R)-enoyl-CoA hydratase with the promoter represented by SEQ ID NO. 17.

In some embodiments of the present invention, the method includes inserting into the genome the gene encoding the PHA polymerase variant capable of synthesizing poly(3-hydroxybutyrate-co-3-hydroxyhexanoate).

In some embodiments of the present invention, the method includes introducing a plasmid carrying the gene encoding the PHA polymerase variant capable of synthesizing poly(3-hydroxybutyrate-co-3-hydroxyhexanoate).

In some embodiments of the present invention, the method includes introducing a plasmid carrying the gene encoding the PHA polymerase variant capable of synthesizing poly(3-hydroxy butyrate-co-3-hydroxyhexanoate) and a proC gene, and inactivating the proC gene in the genome.

Wherein, gene inactivation can be achieved by gene knockout, silent expression, RNA interference and the like.

In a ninth aspect, the present invention provides any one of the following uses of the engineered Ralstonia eutropha:
(1) use in the fermentation production of polyhydroxy fatty acid esters or their derivatives: and
(2) use in breeding strains for the fermentation production of polyhydroxy fatty acid esters or their derivatives.

In the above use, the breeding of strains for fermentation production of polyhydroxy fatty acid esters or their derivatives may specifically be as follows: using the engineered Ralstonia eutropha provided by the present invention as the original strain, breeding the strains for fermentation production of polyhydroxy fatty acid esters or their derivatives by genetic engineering modification, mutagenesis or domestication methods.

In a tenth aspect, the present invention provides a method for fermentative production of polyhydroxy fatty acid esters or derivatives thereof, which includes the step of culturing the engineered Ralstonia eutropha and obtaining a culture.

In some embodiments of the present invention, the method includes the following steps: activating and culturing the engineered Ralstonia eutropha, inoculating the activated bacteria into a seed medium for seed culture to obtain a seed solution, and then inoculating the seed solution into a production medium to obtain the culture.

The medium commonly used for the culture of Ralstonia eutropha can be selected for the above culture. The medium may contain carbon source, nitrogen source and inorganic salt. Among them, the carbon source includes, but is not limited to, a combination of one or more of vegetable oils (e.g. palm oil), sucrose, glucose, molasses, maltose, fructose, and arabinose: the nitrogen source includes, but is not limited to, a combination of one or more of corn syrup, yeast extract, urea, ammonium sulfate, ammonium chloride, ammonium nitrate, and potassium nitrate: the inorganic salt includes, but is not limited to, phosphate, potassium salt, sodium salt, magnesium salt, zinc salt, iron salt, manganese salt, calcium salt, borate, cobalt salt, copper salt, nickel salt and molybdenum salt.

In some embodiments of the present invention, the method further includes the step of separating and extracting the culture obtained from the culture to collect the polyhydroxy fatty acid esters or their derivatives.

In an eleventh aspect, the present invention provides a method for increasing the proportion of 3-hydroxyhexanoic acid in PHA produced by an engineered microorganism, the method including: modifying a Ralstonia eutropha to express an acetoacetyl-CoA reductase variant, and the amino acid sequence of the acetoacetyl-CoA reductase variant is represented by any one of SEQ ID NOs. 9-15.

In the above methods, expression of the acetoacetyl-CoA reductase variant may be achieved by any one or more of the following methods:
(1) introducing a plasmid comprising a gene encoding the acetoacetyl-CoA reductase variant:
(2) inserting one or more copies of the gene encoding the acetoacetyl-CoA reductase variant into the genome.

In some embodiments of the present invention, the gene encoding the acetoacetyl-CoA reductase variant is represented by any one of SEQ ID NOs. 1-7.

In some embodiments of the present invention, the genomic in situ phaB gene of the microorganism is not inactivated.

In some embodiments of the present invention, the genomic in situ phaB gene of the microorganism is retained intact.

In the method described above, the engineered microorganism further includes one or more of the following modifications:
(1) expression of a PHA polymerase variant capable of synthesizing poly(3-hydroxybutyrate-co-3-hydroxyhexanoate);
(2) enhanced expression and/or enzyme activity of (R)-enoyl-CoA hydratase.

In some embodiments of the present invention, the PHA polymerase variant capable of synthesizing poly(3-hydroxybutyrate-co-3-hydroxyhexanoate) contains a mutation of asparagine to serine at position 149 and a mutation of aspartate to glycine at position 171 compared to the original PHA polymerase.

In some embodiments of the present invention, the amino acid sequence of the PHA polymerase variant is represented by SEQ ID NO. 16.

In some embodiments of the present invention, enhanced expression and/or enzyme activity of (R)-enoyl-CoA hydratase is achieved by initiating the transcription of the gene encoding the genome (R)-enoyl-CoA hydratase with the promoter represented by SEQ ID NO. 17.

In some embodiments of the present invention, the increase in the proportion of 3-hydroxyhexanoic acid in PHA produced by the engineered microorganism is an increase in the proportion of 3-hydroxyhexanoic acid in PHA produced by the engineered microorganism using lipids as carbon sources.

In some embodiments of the present invention, the increase in the proportion of 3-hydroxyhexanoic acid in PHA produced by the engineered microorganism is an increase in the proportion of 3-hydroxyhexanoic acid in PHA produced by the engineered microorganism using vegetable oils as carbon sources.

The vegetable oils include a mixture selected from one or more of palm oil, peanut oil, soybean oil, flax oil, rapeseed oil, castor oil, and corn oil.

In the present invention, the polyhydroxy fatty acid ester is preferably poly(3-hydroxy butyrate-co-3-hydroxyhexanoate); more preferably, poly(3-hydroxy butyrate-co-3-hydroxyhexanoate) with a high 3-hydroxyhexanoic acid ratio.

The beneficial effect of the present invention is that the acetoacetyl-CoA reductase variants and their coding genes provided by the present invention can significantly increase the proportion of 3-hydroxyhexanoic acid in PHA produced by the strain; and the proportion of 3-hydroxyhexanoic acid in PHA produced by the engineered Ralstonia eutropha constructed using the acetoacetyl-CoA reductase variants and their coding genes provided by the present invention is significantly increased, which provides new gene and strain resources for the development of engineered strains producing poly(3-hydroxy butyrate-co-3-hydroxyhexanoate) with high 3-hydroxycaproic acid ratio.

### BRIEF DESCRIPTION OF THE DRAWINGS

FIG. 1 shows the molar ratio of 3-hydroxyhexanoic acid in PHA produced by fermentation of each strain in Example 2 of the present invention.

FIG. 2 shows the capacity of each strain in Example 2 of the present invention to produce PHA by fermentation.

FIG. 3 shows the molar ratio of 3-hydroxyhexanoic acid in PHA produced by fermentation of each strain in Example 3 of the present invention.

FIG. 4 shows the ability of each strain in Example 3 of the present invention to produce PHA by fermentation.

FIG. 5 shows the molar ratio of 3-hydroxyhexanoic acid in PHA produced by fermentation of each strain in Example 4 of the present invention.

FIG. 6 shows the ability of each strain in Example 4 of the present invention to produce PHA by fermentation.

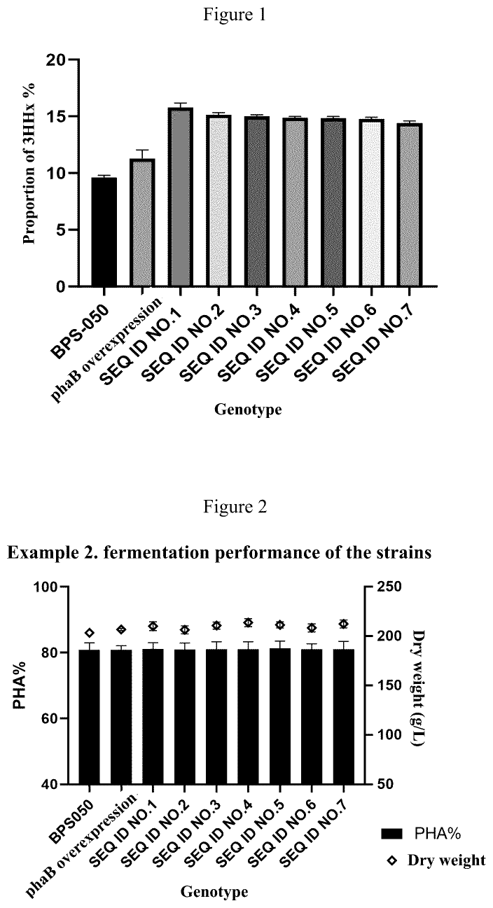

In FIGS. 1, 2, 3, 4, 5 and 6, SEQ ID NO. 1, SEQ ID NO. 2, SEQ ID NO. 3, SEQ ID NO. 4, SEQ ID NO. 5, SEQ ID NO. 6 and SEQ ID NO. 7 represent the engineered Ralstonia eutropha expressing the genes encoding the acetoacetyl-CoA reductase variants with sequences as represented by SEQ ID NO. 1, SEQ ID NO. 2, SEQ ID NO. 3, SEQ ID NO. 4, SEQ ID NO. 5, SEQ ID NO. 6 and SEQ ID NO. 7, respectively.

### Specific Modes for Carrying Out the Embodiments

The application of the present invention is not limited to the embodiments described or exemplified in the specification below: The present invention can be used in other embodiments and may be implemented or carried out in a variety of ways. In addition, the phrases and terms used herein are for descriptive purposes and should not be regarded as limiting. As used herein, the words “include”, “comprise”, or “have”, “contain”, “relate to” and variations thereof are intended to include the items enumerated below and their equivalents, as well as other items.

The specific embodiments provided by the present invention are based in part or in whole on the following findings: the present invention found the acetoacetyl-CoA reductase variants and their coding genes that can significantly increase the proportion of 3-hydroxyhexanoic acid in PHA produced by the strain. The genes encoding these acetoacetyl-CoA reductase variants can be introduced into strains having other genes required for the synthesis of PHA, in particular P(3HB-co-3HHx), which can be used to increase the proportion of 3-hydroxyhexanoic acid in PHA produced by these strains, thus obtaining engineered microorganisms. These engineered microorganisms can be used to produce PHA (particularly P(3HB-co-3HHx)), which in turn improves the ability of existing PHA fermentation strains to produce P(3HB-co-3HHx) with high 3-hydroxycaproic acid ratio. Based on the expression of the acetoacetyl-CoA reductase variant provided by the present invention, other modifications can be made to the engineered microorganisms, and the present invention found that the expression of the acetoacetyl-CoA reductase variant can be at least co-modified with the expression of the PHA polymerase (phaC) variant and the enhanced expression of phaJ, so as to further improve the yield of P(3HB-co-3HHx) with high 3-hydroxycaproic acid ratio.

In some embodiments, the present invention provides an acetoacetyl-CoA reductase variant having the amino acid sequence represented by any one of SEQ ID NOs. 9-15. These variants are capable of significantly increasing the proportion of 3-hydroxyhexanoic acid in PHA produced by the strain.

In some embodiments, the present invention provides a gene encoding the acetoacetyl-CoA reductase variant having the nucleotide sequence represented by any one of SEQ ID NOs. 1-7. These genes are optimized for expression in Ralstonia eutropha.

In some embodiments, the present invention provides an engineered Ralstonia eutropha that expresses the coding gene of acetoacetyl-CoA reductase variant.

In some embodiments, the present invention provides an engineered Ralstonia eutropha with the gene encoding the acetoacetyl-CoA reductase variant described above inserted into the genome.

In some embodiments, the present invention provides an engineered Ralstonia eutropha with the gene encoding the acetoacetyl-CoA reductase variant described above is inserted into the genome of Ralstonia eutropha BPS-050. These engineered Ralstonia eutropha produced a significantly higher proportion of 3-hydroxyhexanoic acid in P(3HB-co-3HHx) compared to Ralstonia eutropha BPS-050.

In some embodiments, the present invention provides an engineered Ralstonia eutropha with the gene encoding the acetoacetyl-CoA reductase variant described above inserted at the phaC gene in the genome of Ralstonia eutropha BPS-050.

In some embodiments, the present invention provides an engineered Ralstonia eutropha with the phaC gene in the genome of Ralstonia eutropha H16 replaced with a gene encoding a PHA polymerase variant (sequence as represented by SEQ ID NO. 16) and with the gene encoding the acetoacetyl-CoA reductase variant described above inserted into their genome. These engineered Ralstonia eutropha produced a significantly higher proportion of 3-hydroxyhexanoic acid in P(3HB-co-3HHx).

In some embodiments, the present invention provides an engineered Ralstonia eutropha with the promoter upstream of the phaJ4b gene of the genome of Ralstonia eutropha BPS-050 replaced with the promoter represented by SEQ ID NO. 17 and with the gene encoding the acetoacetyl-CoA reductase variant described above inserted into their genome. These engineered Ralstonia eutropha produced a significantly higher proportion of 3-hydroxyhexanoic acid in P(3HB-co-3HHx).

The following Examples are used to illustrate the present invention, but are not intended to limit the scope of the invention.

The experimental methods used in the following Examples are conventional if not otherwise specified.

The materials, reagents and the like used in the following Examples are commercially available if not otherwise specified. The enzyme reagents used were purchased from New England Biolabs (NEB) Co., Ltd., the kits for plasmid extraction were purchased from TIANGEN Biotech (Beijing) Co., Ltd., and the kits for DNA fragment recovery were purchased from Omega, USA. The corresponding operation steps were strictly in accordance with the product instructions. All culture mediums were prepared with deionized water unless otherwise specified.

The medium formulations used in the following Examples are as follows:
Seed medium I: 10 g/L peptone, 5 g/L Yeast Extract and 3 g/L glucose.
Seed medium II: 0.15% palm oil, 10 g/L peptone and 5 g/L Yeast Extract.
Production medium: 1.0% palm oil, 9.85 g/L Na₂HPO₄·12H₂O, 1.5 g/L KH₂PO₄, 3.0 g/L NH₄Cl, 10 mL/L trace element solution I and 1 mL/L trace element solution II. Wherein, the composition of trace element solution I includes 20 g/L MgSO₄ and 2 g/L CaCl₂). The composition of trace element solution II includes 100 mg/L ZnSO₄·7H₂O, 30 mg/L MnCl₂·4H₂O, 300 mg/L H₃BO₃, 200 mg/L CoCl₂·6H₂O, 10 mg/L CuSO4.5H₂O, 20 mg/L NiCl₂·6H₂O and 30 mg/L NaMoO₄·2H₂O. The above reagents were all purchased from Sinopharm Chemical Reagent Co., Ltd.

### Example 1: Construction of a Library of Transformants Expressing Acetoacetyl-CoA Reductase Variants Using BPS-050 as an Original Bacterium

In the present Example, Ralstonia eutropha BPS-050 was used as an original bacterium, and the original bacterium was modified to express different acetoacetyl-CoA reductase variants respectively, which specifically included the following steps:

### Step 1: Construction of Basic Plasmids

PCR amplification was performed using the genome of Ralstonia eutropha as a template to obtain the phaC upstream and downstream homologous fragments phaC-H1 and phaC-H2, and the BsaI sites were added to the posterior and anterior ends of phaC-H1 and phaC-H2 to facilitate subsequent operations: the modified plasmid pK18mob (Orita, I., Iwazawa, et al. J. Biosci. Bioeng. 113, 63-69) was used as a template for PCR amplification to obtain the vector fragment: phaC-H1 and phaC-H2 were ligated to the vector fragment by Gibson Assembly method to obtain the recombinant plasmid pKO-C. The primers used were shown in Table 1.

TABLE 1

| Primer | Primer sequence |
|---|---|
| name | (5′-3′) |
| pK-R | gcagacttggccgggtacca |
| | (SEQ ID NO: 18) |
| pK-F | caccgctcgtcacatcctg |
| | (SEQ ID NO: 19) |
| phaCH1-F | tggtacccggccaagtctgc |
| | gggcgtgcccatgatgtaga |
| | (SEQ ID NO: 20) |
| phaCH1-R | tgagacccaaggtctccatg |
| | atttgattgtctctctgccg |
| | tc (SEQ ID NO: 21) |
| phaCH2-F | ggagaccttgggtctcagtg |
| | acgcttgcatgagtgccg |
| | (SEQ ID NO: 22) |
| phaCH2-R | caggatgtgacgagcggtgc |
| | atggtgtcgaccagcttgg |
| | (SEQ ID NO: 23) |

### Step 2: Gene Synthesis

The sequences of the genes encoding different acetoacetyl-CoA reductase variants were optimized separately to make them well expressed in Ralstonia eutropha, and the optimized gene sequences encoding the acetoacetyl-CoA reductase variants are represented by SEQ ID NOs. 1-7.

When the above optimized genes encoding the acetoacetyl-CoA reductase variants were sent to synthesis, GGTCTCATC was added upstream of the DNA sequence and GTGAAGAGACC (SEQ ID NO: 24) was added downstream to facilitate the subsequent operation.

### Step 3: Construction of a Target Strain Containing a Target Gene

The plasmid pKO-C constructed in step 1 was assembled with the plasmid containing the optimized gene encoding the acetoacetyl-CoA reductase variant returned by the gene synthesis company using Goldengate to obtain recombinant plasmids pKO-C-N carrying different genes encoding acetoacetyl-CoA reductase variants, respectively (N stands for the loaded gene encoding the acetoacetyl-CoA reductase variant). Each plasmid was transferred into E. coli S17-1 and then into Ralstonia eutropha BPS-050 by conjugative transfer, and positive clones were screened with LB plates containing both 500 μg/mL spectinomycin and 100 μg/mL apramycin, taking advantage of the inability of the suicide plasmids to replicate in the host bacteria. The recombinant plasmids with homologous fragments in the positive clone were integrated at the specific positions in the genome where H1 and H2 are located, resulting in the first homologous recombinant bacterium.

The first homologous recombinant bacterium was cultured on LB plates containing 100 mg/mL sucrose, and from these monoclonal clones, those without spectinomycin resistance were screened and PCR was performed using primers FphaCH1-F: tggtctggctggcggactgag (SEQ ID NO: 25) and phaCH2-R: ggcgaactcatcctgegcctc (SEQ ID NO: 26). The recombinant bacteria inserted with the target gene were identified by sequencing, and the recombinant bacteria obtained were the stable plasmid version of Ralstonia eutropha ReAproCAphaC::N, with N being the inserted gene.

Step 4: Construction of Recombinant Bacteria Overexpressing the Original phaB Gene of Ralstonia eutropha

Referring to the method for constructing recombinant plasmids in step 3, recombinant plasmids containing the original phaB gene of Ralstonia eutropha were constructed using Gibson assembly as follows:

PCR amplification was performed using the plasmid obtained in step 1 as a template to obtain the plasmid backbone fragment. The phaB gene fragment was obtained by amplification using the genome of Ralstonia eutropha BPS-050 as a template. The above two fragments were ligated by Gibson Assembly method to obtain the recombinant plasmid pKO-C-phaB. The primers used are shown in Table 2.

TABLE 2

| Primer | Primer sequence |
|---|---|
| name | (5′-3′) |
| pKCH-CR | CATGATTTGATTGTCT |
| | CTCTGCCG |
| | (SEQ ID NO: 27) |
| pKCH-CF | GTGACGCTTGCATGAG |
| | TGCC |
| | (SEQ ID NO: 28) |
| phaB F | AGAGAGACAATCAAAT |
| | CATGACTCAGCGCATT |
| | GCGTATG |
| | (SEQ ID NO: 29) |
| phaB R | GGCACTCATGCAAGCG |
| | TCACTCAGCCCATATG |
| | CAGGCCGC |
| | (SEQ ID NO: 30) |

The pKO-C-phaB plasmid was transferred into E. coli S17-1, and the recombinant bacterium was constructed with reference to the method in step 3 above, resulting in the overexpression strain ReAphaC::phaB that integrates the phaB gene at the phaC gene of the genome of Ralstonia eutropha, abbreviated as phaB overexpression.

### Example 2: Fermentation Performance Test of Strains (1)

The fermentation performance of each strain expressing different acetoacetyl-CoA reductase variants was tested using Ralstonia eutropha BPS-050 and overexpression strain ReAphaC::phaB as control bacteria.

First, each strain (1000 μL) constructed in Example 1 preserved in glycerol tube was inoculated in seed medium I (20 mL) for 12 h of primary seed culture; then, 1% of seed medium I was inoculated in seed medium II for secondary seed culture; then 10 v/v % of seed medium II was inoculated into a 2 L small-scale fermentor (T&J Bioengineering Co. Ltd.) filled with 1.1 L production culture medium at 10 v/v %. The operating conditions were 30° C., stirring speed of 800 rpm, aeration rate of 1 L/min, and pH control between 6.7 and 6.8. 28% ammonia solution was used for pH control. Palm oil was continuously used as a carbon source during the incubation, and the incubation time was 54 hours.

The fermentation broth was taken for centrifugation to obtain the bacteria. The bacteria were dried to a constant weight. The weight of the dried bacteria was measured and recorded as dry weight. 100 mL of chloroform was added to the resulting dried bacteria, followed by stirring at room temperature overnight to extract polyester from the bacteria. The bacterial residue was filtered off and concentrated in an evaporator to a total volume of about 30 mL, then about 90 mL of hexane was slowly added and left for 1 hour with slow stirring. The precipitated polyester was filtered out and dried under vacuum at 5CTC for 3 hours. The mass of the dried polyester was measured and the polyester content in the bacterium was calculated.

The results showed that the proportion of PHA of each strain expressing different acetoacetyl-CoA reductase variants was not lower than that of the control bacteria, and the H ratio in PHA produced by each strain expressing different acetoacetyl-CoA reductase variants was significantly higher compared with the control bacteria. The H ratio in PHA produced by each strain is shown in FIG. 1, and the corresponding dry weights and PHA ratios are shown in FIG. 2.

### Example 3: Construction of a Library of Transformants Expressing Acetoacetyl-CoA Reductase Variants Using BPS-050 as an Original Bacterium and the Fermentation Performance Test Thereof

In the present Example, BPS-050 was used as an original bacterium, and the upstream promoter of phaJ4b gene was replaced with phaJ194 (SEQ ID NO.17), and the gene encoding the acetoacetyl-CoA reductase variant (SEQ ID NOs. 1-7) was expressed in it.

Step 1: Replacement of the Promoter Upstream of the phaJ4b Gene of BPS-050 by Homologous Recombination
(1) PCR amplification was performed using the genome of Ralstonia eutropha BPS-050 as a template, and the upstream homologous fragment H1 of the phaJ gene promoter was obtained using phaJ-H1 Fp and phaJ-H1 Rp; the upstream homologous fragment H2 of the phaJ gene promoter was obtained using phaJ-H2 Fp and phaJ-H2 Rp.
(2) Gene synthesis of phaJ194 (SEQ ID NO.17), which is the promoter of phaJ gene
(3) H1 and H2 obtained by PCR and the promoter phaJ194 were ligated with the vector fragment by Gibson Assembly method to obtain the recombinant plasmid pK18mob-phaJ194. The primers used are shown in Table 3.

TABLE 3

| Primer | Primer sequence |
|---|---|
| name | (5′-3′) |
| phaJ-H1 Fp | TGGTACCCGGCCAAGTCTG |
| | TTCGACGGCGTCTTCGTT |
| | (SEQ ID NO: 31) |
| phaJ-H1 Rp | CGAGCGGTGTGGAGGCATC |
| | TATTCAGTCAGGGATGCCT |
| | (SEQ ID NO: 32) |
| phaJ-H2 Fp | CTACAAATAATTTTGTTTA |
| | ACTGACTGAATTCATGGGA |
| | CAAGCATGAAGACCTACGA |
| | GAACA |
| | (SEQ ID NO: 33) |
| phaJ-H2 Rp | CTTGAAGACGAAAGGGCCT |
| | CGTGGCGCCTTATGGAAAT |
| | CAG |
| | (SEQ ID NO: 34) |
| phaJ Fp | ATGCCTCCACACCGCTCG |
| | (SEQ ID NO: 35) |
| phaJ Rp | TTAAACAAAATTATTTGT |
| | AGAGGCTG |
| | (SEQ ID NO: 36) |

(4) The recombinant plasmid pK18mob-pha/194 was transferred into E. coli S17-1 and then into the original bacterium by conjugative transfer, and positive clones were screened with LB plates containing both 200 μg/mL kanamycin and 100 μg/mL apramycin, taking advantage of the inability of the suicide plasmid to replicate in the host bacterium. The recombinant plasmids with homologous fragments in the positive clone were integrated at the specific positions in the genome where H1 and H2 are located, resulting in the first homologous recombinant bacterium.

The first homologous recombinant bacterium was cultured on LB plates containing 100 mg/mL sucrose by scratching monoclonal clones, and from these monoclonal clones, clones without kanamycin resistance were screened and identified by PCR with primers phaJ Fp and phaJ Rp to obtain the recombinant bacterium of the corresponding fragment size, which is Ralstonia eutropha ReΔphaC::phaCac_phaJ194, abbreviated as Re_phaJ194.

Step 2: Different genes (SEQ ID NOs. 1-7) encoding acetoacetyl-CoA reductase variants were inserted into the genome of Ralstonia eutropha ReΔphaC::phaCac_phaJ194, respectively, by the method referring to Example 1.

Step 3: In Ralstonia eutropha ReΔphaC::phaCac_phaJ194, the original phaB gene of Ralstonia eutropha was overexpressed to obtain the phaB overexpression strain phaB overexpression-2.

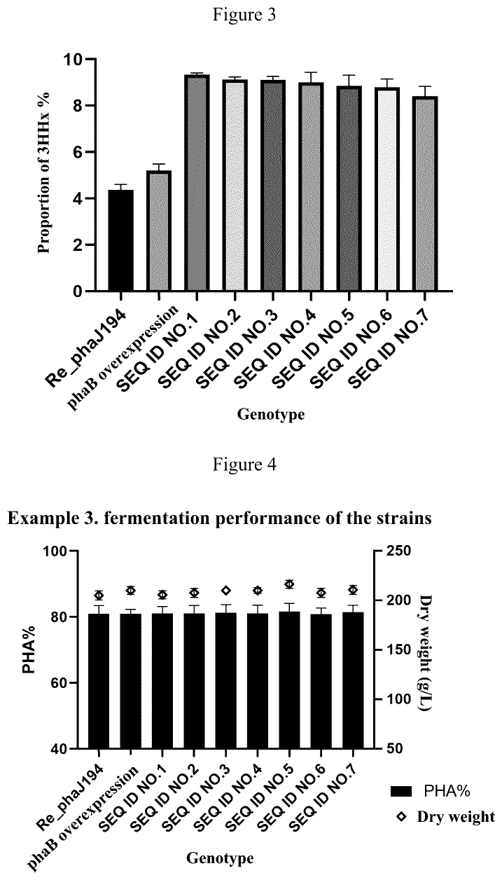

Step 4: The strains constructed in steps 2 and 3 were subjected to fermentation culture and PHA detection according to the method of Example 2. The results showed that the PHA proportion of each strain expressing different acetoacetyl-CoA reductase variants was not lower than that of the control bacteria, and the H ratio in the PHA produced by each strain expressing different acetoacetyl-CoA reductase variants was significantly higher compared with the control bacteria, and the H ratio in the PHA produced by each strain is shown in FIG. 3, and the corresponding dry weights and PHA ratios thereof are shown in FIG. 4.

### Example 4: Construction of a Library of Transformants Expressing Acetoacetyl-CoA Reductase Variants Using H16 as an Original Bacterium and the Fermentation Performance Test Thereof

In the present Example, Ralstonia eutropha H16 was used as an original bacterium, the genomic phaC gene of H16 was mutated to a phaC gene variant (the sequence of the encoded protein is represented by SEQ ID NO. 16), which enabled the recombinant bacteria to acquire the ability to synthesize 3HHx and express the genes encoding the acetoacetyl-CoA reductase variants (SEQ ID NOs. 1-7) in the obtained recombinant bacteria.

Step 1: Replacement of the phaC Gene in the Genome of Ralstonia eutropha

The sequence represented by SEQ ID NO. 8 was obtained by gene synthesis, the sequence carries approximately 600 bp upstream and downstream of the phaC gene and the phaC gene variant. Plasmid construction was performed by using the plasmids constructed in step 1 of Example 1, GGTCTCATC was added upstream and GTGAAGAGACC was added downstream of the DNA sequence during gene synthesis. A recombinant plasmid pK18mob-ΔphaC::phaCac was obtained by ligating the synthesized gene to the vector fragment via the Goldengate method.

The recombinant plasmid pK18mob-ΔphaC::phaCac was transferred into E. coli S17-1 and then into Ralstonia eutropha by conjugative transfer method, and positive clones were screened with LB plates containing both 200 μg/mL kanamycin and 100 μg/mL apramycin, taking advantage of the inability of the suicide plasmids to replicate in the host bacteria. The recombinant plasmids with homologous fragments in the positive clone were integrated into the genome at the specific locations where H1 and H2 are located, resulting in the first homologous recombinant bacterium.

The first homologous recombinant bacterium was cultured on LB plates containing 100 mg/mL sucrose by scratching monoclonal clones, and from these monoclonal clones, clones without kanamycin resistance were screened and PCR was performed with primers phaC-H1 FP and phaC-H2 RP, and the recombinant bacterium with phaC gene substitution was sequenced to identify the recombinant bacterium, which was obtained as Ralstonia eutropha ReΔphaC::phaCac, abbreviated as Re_H16.

Step 2: Different genes (SEQ ID NOs. 1-7) encoding acetoacetyl-CoA reductase variants were inserted into the genome of the Ralstonia eutropha ReΔphaC::phaCac constructed in step 1, respectively, by the method referring to Example 1.

Step 3: In Ralstonia eutropha ReΔphaC::phaCac constructed in step 1, the original phaB gene of Ralstonia eutropha H16 was overexpressed to obtain the phaB overexpression strain phaB overexpression-3.

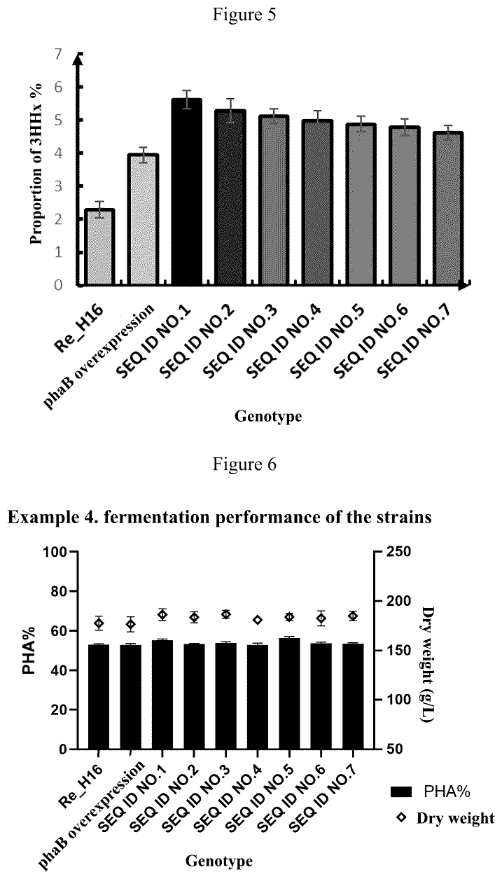

Step 4: The strains constructed in steps 2 and 3 were subjected to fermentation culture and PHA detection according to the method of Example 2. The results showed that the PHA proportion of each strain expressing different acetoacetyl-CoA reductase variants was not lower than that of the control bacteria, and the H ratio in the PHA produced by each strain expressing different acetoacetyl-CoA reductase variants was significantly higher compared with the control bacteria, and the H ratio in the PHA produced by each strain is shown in FIG. 5, and the corresponding dry weights and PHA ratios thereof are shown in FIG. 6.

### Example 5: Fermentation Performance Test of Strains (2)

The strains constructed in Examples 1, 3 and 4 above were subjected to fermentation experiments using other vegetable oils as carbon sources, i.e., the carbon sources in their corresponding methods of fermentation test were replaced from palm oil to soybean oil and flax oil, respectively, and the fermentation performance tests were conducted for each strain constructed in Examples 1, 3 and 4. The results showed that the H ratio in PHA production and the ratio of cell dry weight to PHA for each strain when soy bean oil or flax oil was used as the carbon source tended to be consistent with the results of the fermentation performance tests performed with palm oil as the carbon source, showing no significant differences.

Although, the present invention has been described in detail above with a general description and specific embodiments, some modifications or improvements can be made on the basis of the present invention, as will be apparent to a person skilled in the art. Therefore, these modifications or improvements made without departing from the spirit of the present invention are within the scope of protection claimed by the present invention.

### INDUSTRIAL APPLICABILITY

The present invention provides engineered microorganisms expressing acetoacetyl-CoA reductase variants and their coding genes, uses of the acetoacetyl-CoA reductase variants and their coding genes in increasing the proportion of 3-hydroxyhexanoic acid in PHA produced by microorganisms, and methods for increasing the proportion of 3-hydroxyhexanoic acid in PHA produced by microorganisms. By expressing the acetoacetyl-CoA reductase variant in microorganisms, the proportion of 3-hydroxyhexanoic acid in PHA synthesized by microorganisms is significantly increased, and at the same time, a high content of PHA in microbial cells can be ensured. The acetoacetyl-CoA reductase variants, their coding genes and the engineered microorganisms provided by the present invention provide new genes and strain resources for the development of the strains producing PHA with high proportion of 3-hydroxyhexanoic acid, and have important economic value and application prospects for the production of PHA with high proportion of 3-hydroxyhexanoic acid.

1-3. (canceled)

4. An engineered Ralstonia eutropha, wherein the engineered Ralstonia eutropha expresses an acetoacetyl-CoA reductase variant, the acetoacetyl-CoA reductase variant having an amino acid sequence as represented by any one of SEQ ID NO. 10.

5. The engineered Ralstonia eutropha according to claim 4, wherein the expression of the acetoacetyl-CoA reductase variant is achieved by any one or more of the following ways:
(1) introducing a plasmid comprising a gene encoding the acetoacetyl-CoA reductase variant; and
(2) inserting one or more copies of the gene encoding the acetoacetyl-CoA reductase variant into a genome of the engineered Ralstonia eutropha.

6. The engineered Ralstonia eutropha according to claim 5, wherein the gene encoding the acetoacetyl-CoA reductase variant is represented by any one of SEQ ID NO. 2.

7. The engineered Ralstonia eutropha according to claim 4, wherein the engineered Ralstonia eutropha further comprises one or more of the following modifications:
(1) expression of a polyhydroxyalkanoate polymerase variant having an amino acid sequence of SEQ ID NO. 16, capable of synthesizing poly(3-hydroxybutyrate-co-3-hydroxyhexanoate); and
(2) enhanced expression and/or enzyme activity of (R)-enoyl-CoA hydratase, wherein the enhanced expression and/or enzyme activity of the (R)-enoyl-CoA hydratase is achieved by initiating transcription of a gene encoding (R)-enoyl-CoA hydratase in the genome with a promoter represented by SEQ ID NO. 17.

8. (canceled)

9. A method for constructing the engineered Ralstonia eutropha according to claim 4, wherein the method comprises a step of modifying the Ralstonia eutropha to express the acetoacetyl-CoA reductase variant.

10. (canceled)

11. A method for constructing the engineered Ralstonia eutropha according to claim 5, wherein the method comprises a step of modifying the Ralstonia eutropha to express the acetoacetyl-CoA reductase variant.

12. A method for constructing the engineered Ralstonia eutropha according to claim 6, wherein the method comprises a step of modifying the Ralstonia eutropha to express the acetoacetyl-CoA reductase variant.

13. A method for constructing the engineered Ralstonia eutropha according to claim 7, wherein the method comprises a step of modifying the Ralstonia eutropha to express the acetoacetyl-CoA reductase variant.

14. The engineered Ralstonia eutropha according to claim 5, wherein the engineered Ralstonia eutropha further comprises one or more of the following modifications:
(1) expression of a polyhydroxyalkanoate polymerase variant having an amino acid sequence of SEQ ID NO. 16, capable of synthesizing poly(3-hydroxybutyrate-co-3-hydroxyhexanoate); and
(2) enhanced expression and/or enzyme activity of (R)-enoyl-CoA hydratase, wherein the enhanced expression and/or enzyme activity of the (R)-enoyl-CoA hydratase is achieved by initiating transcription of a gene encoding (R)-enoyl-CoA hydratase in the genome with a promoter represented by SEQ ID NO. 17.

15. The engineered Ralstonia eutropha according to claim 6, wherein the engineered Ralstonia eutropha further comprises one or more of the following modifications:
(1) expression of a polyhydroxyalkanoate polymerase variant having an amino acid sequence of SEQ ID NO. 16, capable of synthesizing poly(3-hydroxybutyrate-co-3-hydroxyhexanoate); and
(2) enhanced expression and/or enzyme activity of (R)-enoyl-CoA hydratase, wherein the enhanced expression and/or enzyme activity of the (R)-enoyl-CoA hydratase is achieved by initiating transcription of a gene encoding (R)-enoyl-CoA hydratase in a genome of the engineered Ralstonia eutropha with a promoter represented by SEQ ID NO. 17.

16. A method for constructing the engineered Ralstonia eutropha according to claim 14, wherein the method comprises a step of modifying the Ralstonia eutropha to express the acetoacetyl-CoA reductase variant.

17. A method for constructing the engineered Ralstonia eutropha according to claim 15, wherein the method comprises a step of modifying the Ralstonia eutropha to express the acetoacetyl-CoA reductase variant.
